SEQUENCE LISTING (77 sequences)

SEQ ID NO: 1 - protein, 35 aa, Mus musculus
RLAGLLRKGGEKIGEKLKKIGKIKNFFQKLVPQPE

SEQ ID NO: 2 - protein, 11 aa, Artificial
  Synthetic peptide
KLKLLLLLKLK

SEQ ID NO: 3 - protein, 29 aa, Artificial
  Synthetic peptide
KFPGGGQIVGGVYLLPRRGPRLGVRATRK

SEQ ID NO: 4 - protein, 14 aa, Artificial
  Synthetic peptide
GYKVLVLNPSVAAT

SEQ ID NO: 5 - protein, 19 aa, Artificial
  Synthetic peptide
AYAAQGYKVLVLNPSVAAT

SEQ ID NO: 6 - protein, 13 aa, Artificial
  Synthetic peptide
  Xaa (8)..(8): Ala or Lys
DLMGYIPXVGAPL

SEQ ID NO: 7 - protein, 25 aa, Artificial
  Synthetic peptide
GEVQVVSTATQSFLATCINGVCWTV

SEQ ID NO: 8 - protein, 23 aa, Artificial
  Synthetic peptide
HMWNFISGIQYLAGLSTLPGNPA

SEQ ID NO: 9 - protein, 32 aa, Artificial
  Synthetic peptide
  Xaa (14)..(14): Val or Ile
  Xaa (16)..(16): Phe or Tyr
  Xaa (21)..(21): Val or Ile
VDYPYRLWHYPCTXNXTIFKXRMYVGGVEHRL

SEQ ID NO: 10 - protein, 16 aa, Artificial
  Synthetic peptide
AAWYELTPAETTVRLR

SEQ ID NO: 11 - protein, 25 aa, Artificial
  Synthetic peptide
GQGWRLLAPITAYSQQTRGLLGCIV

SEQ ID NO: 12 - protein, 26 aa, Artificial
  Synthetic peptide
IGLGKVLVDILAGYGAGVAGALVAFK

SEQ ID NO: 13 - protein, 16 aa, Artificial
  Synthetic peptide
FTDNSSPPAVPQTFQV

SEQ ID NO: 14 - protein, 19 aa, Artificial
  Synthetic peptide
LEDRDRSELSPLLLSTTEW

SEQ ID NO: 15 - protein, 21 aa, Artificial
  Synthetic peptide
YLVAYQATVCARAQAPPPSWD

SEQ ID NO: 16 - protein, 17 aa, Artificial
  Synthetic peptide
MSTNPKPQRKTKRNTNR

SEQ ID NO: 17 - protein, 21 aa, Artificial
  Synthetic peptide
LINTNGSWHINRTALNCNDSL

SEQ ID NO: 18 - protein, 15 aa, Artificial
  Synthetic peptide
TTILGIGTVLDQAET

SEQ ID NO: 19 - protein, 18 aa, Artificial
  Synthetic peptide
  Xaa (4)..(4): Ser or Val
  Xaa (14)..(14): Ala or Cys
FDSXVLCECYDAGXAWYE

SEQ ID NO: 20 - protein, 20 aa, Artificial
  Synthetic peptide
ARLIVFPDLGVRVCEKMALY

SEQ ID NO: 21 - protein, 15 aa, Artificial
  Synthetic peptide
AFCSAMYVGDLCGSV

SEQ ID NO: 22 - protein, 15 aa, Artificial
  Synthetic peptide
GVLFGLAYFSMVGNW

SEQ ID NO: 23 - protein, 18 aa, Artificial
  Synthetic peptide
VVCCSMSYTWTGALITPC

SEQ ID NO: 24 - protein, 15 aa, Artificial
  Synthetic peptide
TRVPYFVRAQGLIRA

SEQ ID NO: 25 - protein, 15 aa, Artificial
  Synthetic peptide
TTLLFNILGGWVAAQ

SEQ ID NO: 26 - protein, 10 aa, Artificial
  Synthetic peptide
AYAAQGYKVL

SEQ ID NO: 27 - protein, 9 aa, Artificial
  Synthetic peptide
CINGVCWTV

SEQ ID NO: 28 - protein, 20 aa, Artificial
  Synthetic peptide
MWNFISGIQYLAGLSTLPGN

SEQ ID NO: 29 - protein, 10 aa, Artificial
  Synthetic peptide
YLLPRRGPRL

SEQ ID NO: 30 - protein, 8 aa, Artificial
  Synthetic peptide
LPRRGPRL

SEQ ID NO: 31 - protein, 9 aa, Artificial
  Synthetic peptide
GPRLGVRAT

SEQ ID NO: 32 - protein, 9 aa, Artificial
  Synthetic peptide
RLGVRATRK

SEQ ID NO: 33 - protein, 9 aa, Artificial
  Synthetic peptide
DLMGYIPAV

SEQ ID NO: 34 - protein, 10 aa, Artificial
  Synthetic peptide
GYIPLVGAPL

SEQ ID NO: 35 - protein, 13 aa, Artificial
  Synthetic peptide
DLMGYIPLVGAPL

SEQ ID NO: 36 - protein, 19 aa, Artificial
  Synthetic peptide
LEDRDRSELSPLLLSTTEW

SEQ ID NO: 37 - protein, 20 aa, Artificial
  Synthetic peptide
DYPYRLWHYPCTVNFTIFKV

SEQ ID NO: 38 - skipped

SEQ ID NO: 39 - protein, 12 aa, Artificial
  Synthetic peptide
TAYSQQTRGLLG

SEQ ID NO: 40 - protein, 13 aa, Artificial
  Synthetic peptide
SMSYTWTGALITP

SEQ ID NO: 41 - protein, 9 aa, Artificial
  Synthetic peptide
  misc_feature (1)..(1): Xaa can be any naturally occurring amino acid
  misc_feature (3)..(3): Xaa can be any naturally occurring amino acid
  misc_feature (7)..(7): Xaa can be any naturally occurring amino acid
  misc_feature (9)..(9): Xaa can be any naturally occurring amino acid
XZXZZZXZX

SEQ ID NO: 42 - protein, 10 aa, Artificial
  Synthetic peptide
  misc_feature (1)..(1): Xaa can be any naturally occurring amino acid
  misc_feature (3)..(3): Xaa can be any naturally occurring amino acid
  misc_feature (8)..(8): Xaa can be any naturally occurring amino acid
  misc_feature (10)..(10): Xaa can be any naturally occurring amino acid
XZXZZZZXZX

SEQ ID NO: 43 - protein, 11 aa, Artificial
  Synthetic peptide
  misc_feature (1)..(1): Xaa can be any naturally occurring amino acid
  misc_feature (3)..(3): Xaa can be any naturally occurring amino acid
  misc_feature (9)..(9): Xaa can be any naturally occurring amino acid
  misc_feature (11)..(11): Xaa can be any naturally occurring amino acid
XZXZZZZZXZX

SEQ ID NO: 44 - protein, 12 aa, Artificial
  Synthetic peptide
  misc_feature (1)..(1): Xaa can be any naturally occurring amino acid
  misc_feature (3)..(3): Xaa can be any naturally occurring amino acid
  misc_feature (10)..(10): Xaa can be any naturally occurring amino acid
  misc_feature (12)..(12): Xaa can be any naturally occurring amino acid
XZXZZZZZZXZX

SEQ ID NO: 45 - protein, 13 aa, Artificial
  Synthetic peptide
  misc_feature (1)..(1): Xaa can be any naturally occurring amino acid
  misc_feature (3)..(3): Xaa can be any naturally occurring amino acid
  misc_feature (11)..(11): Xaa can be any naturally occurring amino acid
  misc_feature (13)..(13): Xaa can be any naturally occurring amino acid
XZXZZZZZZZXZX

SEQ ID NO: 46 - protein, 36 aa, Artificial
  Synthetic peptide
RLAGLLRKGGEKIGEKLKKIGQKIKNFFQKLVPQPE

SEQ ID NO: 47 - protein, 11 aa, Artificial
  Synthetic peptide
KLKLLLLLKLK

SEQ ID NO: 48 - protein, 16 aa, Artificial
  Synthetic peptide
GEVQVVSTATQSFLAT

SEQ ID NO: 49 - protein, 20 aa, Artificial
  Synthetic peptide
NFISGIQYLAGLSTLPGNPA

SEQ ID NO: 50 - protein, 9 aa, Artificial
  Synthetic peptide
QYLAGLSTL

SEQ ID NO: 51 - protein, 9 aa, Artificial
  Synthetic peptide
HMWNFISGI

SEQ ID NO: 52 - protein, 22 aa, Artificial
  Synthetic peptide
KFPGGGQIVGGVYLLPRRGPRL

SEQ ID NO: 53 - protein, 17 aa, Artificial
  Synthetic peptide
YLLPRRGPRLGVRATRK

SEQ ID NO: 54 - protein, 15 aa, Artificial
  Synthetic peptide
LPRRGPRLGVRATRK

SEQ ID NO: 55 - protein, 11 aa, Artificial
  Synthetic peptide
GPRLGVRATRK

SEQ ID NO: 56 - protein, 22 aa, Artificial
  Synthetic peptide
KFPGGYLLPRRGPRLGVRATRK

SEQ ID NO: 57 - protein, 17 aa, Artificial
  Synthetic peptide
AAQGYKVLVLNPSVAAT

SEQ ID NO: 58 - protein, 12 aa, Artificial
  Synthetic peptide
KVLVLNPSVAAT

SEQ ID NO: 59 - protein, 16 aa, Artificial
  Synthetic peptide
AYAAQGYKVLVLNPSV

SEQ ID NO: 60 - protein, 18 aa, Artificial
  Synthetic peptide
AYAAQGYKVLVLNPSVAA

SEQ ID NO: 61 - protein, 15 aa, Artificial
  Synthetic peptide
AAQGYKVLVLNPSVA

SEQ ID NO: 62 - protein, 16 aa, Artificial
  Synthetic peptide
AYAAQGYKVLPSVAAT

SEQ ID NO: 63 - protein, 13 aa, Artificial
  Synthetic peptide
AYAAQGYKVLAAT

SEQ ID NO: 64 - protein, 14 aa, Artificial
  Synthetic peptide
DLMGYIPALVGAPL

SEQ ID NO: 65 - protein, 10 aa, Artificial
  Synthetic peptide
  Xaa (8)..(8): Ala or Lys
DLMGYIPXVG

SEQ ID NO: 66 - protein, 12 aa, Artificial
  Synthetic peptide
  Xaa (8)..(8): Ala or Lys
DLMGYIPXVGAP

SEQ ID NO: 67 - protein, 9 aa, Artificial
  Synthetic peptide
  Xaa (8)..(8): Ala or Lys
DLMGYIPXV

SEQ ID NO: 68 - protein, 11 aa, Artificial
  Synthetic peptide
DLMGYIPLVGA

SEQ ID NO: 69 - protein, 9 aa, Artificial
  Synthetic peptide
DLMGYIPLV

SEQ ID NO: 70 - protein, 20 aa, Artificial
  Synthetic peptide
VSTATQSFLATCINGVCWTV

SEQ ID NO: 71 - protein, 16 aa, Artificial
  Synthetic peptide
TQSFLATCINGVCWTV

SEQ ID NO: 72 - protein, 20 aa, Artificial
  Synthetic peptide
GEVQVVSTATQSFLATCING

SEQ ID NO: 73 - protein, 22 aa, Artificial
  Synthetic peptide
MWNFISGIQYLAGLSTLPGNPA

SEQ ID NO: 74 - protein, 18 aa, Artificial
  Synthetic peptide
NFISGIQYLAGLSTLPGN

SEQ ID NO: 75 - protein, 18 aa, Artificial
  Synthetic peptide
HMWNFISGIQYLAGLSTL

SEQ ID NO: 76 - protein, 17 aa, Artificial
  Synthetic peptide
HMWNFISGISTLPGNPA

SEQ ID NO: 77 - protein, 17 aa, Artificial
  Synthetic peptide
HMWQYLAGLSTLPGNPA

(19) United States
(12) Patent Application Publication
(10) Pub. No.: US 2009/0186047 A1
(43) Pub. Date: Jul. 23, 2009

(54) HCV Vaccinations

(75) Inventors: Alexander Von Gabain, Vienna (AT); Katherine Cohen, Vienna (AT); Karen Lingnau, Vienna (AT); Michael Ginzler, Vienna (AT); Erich Tauber, Muckendorf a.d. Donau (AT); Christoph Klade, Wiener Neustadt (AT); Alessandra Formica, Vienna (AT); Wolfgang Zauner, Vienna (AT)

Correspondence Address: FULBRIGHT & JAWORSKI L.L.P., 600 CONGRESS AVE., SUITE 2400, AUSTIN, TX, 78701, US

(73) Assignee: INTERCELL AG, Vienna (AT)

(21) Appl. No.: 12/298,509

(22) PCT Filed: Apr. 25, 2006
(86) PCT No.: PCT/AT2006/000166

§ 371 Date: Oct. 24, 2008

(51) Int. Cl. A61K 39/29

(52) U.S. Cl. 424/189.1; 424/228.1

(57) ABSTRACT

The invention relates to a method for preventing or treating Hepatitis C Virus (HCVi) infections, wherein a HCV vaccine comprising an effective amount of at least one HCV T-cell antigen and a polycationic compound comprising peptide bonds is administered to a human individual bi-weekly at least 3 times.

### CROSS-REFERENCE TO RELATED APPLICATIONS

This application is a national phase application under 35 U.S.C. § 371 of International Application No. PCT/AT2006/000166 filed 25 Apr. 2006. The entire text of the above-referenced disclosure is specifically incorporated herein by reference without disclaimer.

### BACKGROUND OF THE INVENTION

1. Field of the Invention

The present invention relates to vaccines and vaccination strategies for preventing HCV infections and for treating patients with HCV infections, especially patients with chronic hepatitis.

2. Description of Related Art

Chronic hepatitis C virus (HCV) infection is present in approximately 3% of the world's population (about 170 million people). Hepatitis C Virus (HCV) is a member of the flaviviridiae. There are at least 6 HCV genotypes and more than 50 subtypes have been described. In America, Europe and Japan genotypes 1, 2 and 3 are most common. The geographic distribution of HCV genotypes varies greatly with genotype 1a being predominant in the USA and parts of Western Europe, whereas 1b predominates in Southern and Central Europe. HCV is transmitted through the parenteral or percutan route, and replicates in hepatocytes. About 15% of patients experience acute self-limited hepatitis associated with viral clearance and recovery. About 85% of infected persons become chronic carriers. Infection often persists asymptomatically with slow progression for years, however ultimately HCV is a major cause of cirrhosis, end-stage liver disease and liver cancer. Strength and quality of both CD4+ helper T-cell (HTL) and CD8+ cytotoxic T cell (CTL) responses determine whether patients recover (spontaneously or as a consequence of therapy) or develop chronic infection. During the natural course of hepatitis C, liver cirrhosis develops in about 25% of patients and hepatocellular carcinoma in about 5% within 20-30 years. Substantial costs result from treatment of these sequelae of chronic hepatitis C, including liver transplantation.

Combination treatment based on interferon-alpha and ribavirin is currently the standard treatment of patients with chronic hepatitis C. However, a sustained response (SR) to treatment—as defined by lack of detectable viremia 6 months after cessation of treatment—is achieved in about 50% of patients, and only in 43 to 46% of patients infected with genotype 1, which is the most prevalent in Europe, USA and Canada. The low tolerability and the considerable side effects of this therapy clearly necessitate novel therapeutic intervention including therapeutic vaccines. Evaluation of new treatment modalities is therefore warranted.

Interferon-alpha based therapies have substantial side effects, such as flu-like syndrome, fever, headache, arthralgia, myalgia, depression, weight loss, alopecia, leukopenia, and thrombocytopenia. These side effects are frequently quite marked and may limit quality of life or the ability to work. Interferon treatment is limited especially by the hematologic side effects (thrombocytopenia) and is contraindicated in many patients with pre-existing thrombocytopenia due to liver cirrhosis with splenomegaly.

Ribavirin also has several side effects that may be clinically significant. Ribavirin induces haemolysis and significant anaemia that may result in decreased oxygen delivery to tissues and has been associated with myocardial infarction in patients with coronary heart disease. In addition, administration of ribavirin is potentially teratogenic, mutagenic, and carcinogenic. Anticonceptive measures are therefore mandatory during ribavirin therapy in fertile male and female patients.

Other possible treatment strategies, such as HCV-specific protease, polymerase or helicase inhibitors, are still in the pre-clinical phase or early clinical development. Their clinical availability cannot be foreseen at present.

Because of this limited efficacy of standard treatment on the one hand and important side-effects on the other hand, new treatment modalities for hepatitis C are urgently needed.

A strategy which has been pursued aims at the development of peptide based vaccines. Approaches which have already shown that this route can be successful are described e.g. in WO 01/24822, WO 2004/024182, WO 2005/004910 or PCT/EP2005/054773.

### SUMMARY OF THE INVENTION

Therefore, the present invention relates to a method for preventing or treating Hepatitis C Virus (HCV) infections, wherein a HCV vaccine comprising
an effective amount of at least one HCV T-cell antigen and
a polycationic compound comprising peptide bonds is administered to a human individual bi-weekly at least 3 times.

According to the present invention, it has surprisingly turned out that efficacy of HCV vaccines containing HCV T-cell antigens are highly dependent on the administration rate. Other administration parameters, such as route of administration, total number of vaccine doses or amount of antigen applied per dose, are also important, but not as critical for optimal efficacy as administration rate. An efficient administration rate should reflect the balance between vaccination response and burden for the human individual to be vaccinated. According to the present invention the bi-weekly administration of an HCV T-cell vaccine turned out to be superior in overall efficacy compared to e.g. daily, weekly or monthly (four-weekly) administration. This could be demonstrated by comparative clinical trials both, in healthy volunteers and also in patients, especially chronic HCV patients.

According to the present invention it is preferred to keep the bi-weekly administration as strict as possible to the 14 days interval. However, also the administration of the vaccine in intervals of 10 to 20 days, preferably 11 to 18 days, especially 12 to 16 days (which could be necessitated by practical circumstances such as availability and health status of the patient), is—due to the standard practice for such vaccination strategies—still considered as meeting the requirement of “bi-weekly” administration.

Although efficacy of vaccination is not excluded by two times or three times bi-weekly administration, it is preferred that the HCV vaccine according to the present invention is administered bi-weekly at least 4 times, preferably at least 6 times, especially at least 8 times. Such an at least 12 to 16 week vaccination strategy has proven to be specifically effective for chronic HCV patients. It is also possible to apply an interrupted vaccination strategy e.g. with boostering injections after a longer break after the initial vaccination. For example, after a first vaccination phase with 3, 4, 5, 6, 7 or 8 bi-weekly vaccinations will be followed by a booster later, e.g. one to twelve, preferably two to six months after the bi-weekly vaccinations.

It is preferred to combine the HCV T-cell epitopes in the HCV vaccine according to the present invention with suitable adjuvants, immunostimulatory substances, etc. in order to enhance or assure suitable presentation of the HCV T-cell antigens to the immune system of the individual to whom the vaccine should be administered. Therefore, the vaccine according to the present invention comprises—in addition to the HCV antigens—a polycationic compound comprising peptide bonds. Preferably, the polycationic compound comprising peptide bonds according to the present invention is selected from the group consisting of basic polypeptides, organic polycations, basic polyamino acids and mixtures thereof. Preferred polycationic compounds comprising peptide bonds comprise a peptide chain having a chain length of at least 4 amino acid residues.

Accordingly, polycationic compounds are preferred which are selected from the group consisting of polypeptides containing more than 20%, especially more than 50% of basic amino acids in a range of more than 8, especially more than 20, amino acid residues, especially polyarginine or polylysine, polycationic antimicrobial peptides, peptide containing at least 2 KLK-motifs separated by a linker of 3 to 7 hydrophobic amino acids, or mixtures thereof. Preferably, the polycationic compound comprising peptide bonds according to the present invention contains between 20 and 500 amino acid residues, especially between 30 and 200 residues.

These polycationic compounds may be produced chemically or recombinantly or may be derived from natural sources.

Cationic (poly)peptides may also be anti-microbial peptides. These (poly)peptides may be of prokaryotic or animal or plant origin or may be produced chemically or recombinantly. Peptides may also belong to the class of defensins. Sequences of such peptides can, for example, be found in suitable review articles (e.g. Curr Pharm Des. 2002; 8(9):743-61) or in the Antimicrobial Sequences Database under the following internet address:
http://www.bbcm.univ.trieste.it/˜tossi/pag2.html.

Such host defence peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substance in the present invention are cathelicidin derived antimicrobial peptides or derivatives thereof (International patent application WO 02/13857, incorporated herein by reference), especially antimicrobial peptides derived from mammal cathelicidin, preferably from human, bovine or mouse.

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide which has the amino acid sequence NH2-RLAGLLRKGGEKIGEKLKKIGQKIKNFFQKLVPQPE-COOH (SEQ ID NO:46). Related or derived cathelin substances contain the whole or parts of the cathelin sequence with at least 15-20 amino acid residues. Derivations may include the substitution or modification of the natural amino acids by amino acids which are not among the 20 standard amino acids. Moreover, further cationic residues may be introduced into such cathelin molecules. These cathelin molecules are preferred to be combined with the antigen. These cathelin molecules surprisingly have turned out to be also effective as an adjuvant for an antigen without the addition of further adjuvants. It is therefore possible to use polycationic compounds comprising peptide bonds according to the present invention, e.g. such cathelin molecules as efficient adjuvants in vaccine formulations with or without further immunoactivating substances.

Another preferred polycationic substance to be used according to the present invention is a synthetic peptide containing at least 2 KLK-motifs separated by a linker of 3 to 7 hydrophobic amino acids (International patent application WO 02/32451, incorporated herein by reference). Therefore, a preferred HCV vaccine further contains a peptide comprising a sequence R1-XZXZNXZX-R2 (SEQ ID NOS:41-45, wherein: xzxzzzxzx=SEQ ID NO:41; xzxzzzzxzx=SEQ ID NO:42; xzxzzzzzxzx=SEQ ID NO:43; xzxzzzzzzxzx=SEQ ID NO:44; and xzxzzzzzzzxzx=SEQ ID NO:45), whereby N is a whole number between 3 and 7, preferably 5, X is a positively charged natural and/or non-natural amino acid residue, Z is an amino acid residue selected from the group consisting of L, V, I, F and/or W, and R1 and R2 are selected independently one from the other from the group consisting of —H, —NH2, —COCH3, —COH, a peptide with up to 20 amino acid residues or a peptide reactive group or a peptide linker with or without a peptide; X-R2 may be an amide, ester or thioester of the C-terminal amino acid residue of the peptide.

The polycationic substances according to the present invention may also be combined with other immunisers. Preferred examples for such further immunisers are disclosed in WO 01/93905 and WO 02/095027 (I- or U-containing oligodeoxynucleotides (I- or U-ODNs); I-ODNs are also specifically useable as TLR ligands or agonists according to the present invention (see below)).

Preferably, the I- or U-ODNs are combined with the molecules according to WO 02/32451 (especially KLKLLLLLKLK (SEQ ID NO:47)) or polyarginine.

The HCV T-cell antigens to be used according to the present invention should be T-cell antigens from conserved regions of HCV proteins. Therefore, preferably conserved peptide epitopes derived from HCV proteins are used, which are known to be targets of productive immune responses in patients. In order to minimize viral escape, a pool of peptides conserved in the most prevalent strains should preferably be employed. This safeguards induction of HCV specific T-cell immunity. Peptides are recognized by the T-cell receptor in conjunction with MHC molecules. Since HLA-A2 is the most prevalent MHC molecule in Caucasians, in case of MHC class I, only peptides interacting with this HLA allele were chosen. Consequently, for a HCV vaccine which should have an optimum efficacy in this group of population, individuals positive for certain HLA-types, e.g. HLA-A2, should be vaccinated according to the present invention with T-cell epitopes specific for this HLA-type. The length of the HCV T-cell antigens to be used in the present invention is not that critical. Optimisation should take into consideration the peptide synthesis required, solubility, number of T-cell epitopes per polypeptide, etc.

Preferably, the HCV T-cell epitope is provided as a polypeptide consisting of from 7 to 50 amino acid residues, preferably from 8 to 45 amino acid residues, especially from 8 to 20 amino acid residues, each of the peptides comprising at least one T-cell epitope.

Preferred HCV T-cell antigens to be used according to the present invention may be selected from those disclosed as efficient epitopes in WO 01/24822, WO 2004/024182, WO 2005/004910 and/or PCT/EP2005/054773. Preferably, the T-cell antigens are selected from the group consisting of

(SEQ ID NO: 3)

KFPGGGQIVGGVYLLPRRGPRLGVRATRK,

(SEQ ID NO: 4)

GYKVLVLNPSVAAT,

(SEQ ID NO: 5)

AYAAQGYKVLVLNPSVAAT,

(SEQ ID NO: 6)

DLMGYIP(A/L)VGAPL,

(SEQ ID NO: 7)

GEVQVVSTATQSFLATCINGVCWTV,

(SEQ ID NO: 8)

HMWNFISGIQYLAGLSTLPGNPA,

(SEQ ID NO: 9)

VDYPYRLWHYPCT(V/I)N(F/Y)TIFK(V/I)RMYVGGVEHRL,

(SEQ ID NO: 10)

AAWYELTPAETTVRLR,

(SEQ ID NO: 11)

GQGWRLLAPITAYSQQTRGLLGCIV,

(SEQ ID NO: 12)

IGLGKVLVDILAGYGAGVAGALVAFK,

(SEQ ID NO: 13)

FTDNSSPPAVPQTFQV,

(SEQ ID NO: 14)

LEDRDRSELSPLLLSTTEW,

(SEQ ID NO: 15)

YLVAYQATVCARAQAPPPSWD,

(SEQ ID NO: 16)

MSTNPKPQRKTKRNTNR,

(SEQ ID NO: 17)

LINTNGSWHINRTALNCNDSL,

(SEQ ID NO: 18)

TTILGIGTVLDQAET,

(SEQ ID NO: 19)

FDS(S/V)VLCECYDAG(A/C)AWYE,

(SEQ ID NO: 20)

ARLIVFPDLGVRVCEKMALY,

(SEQ ID NO: 21)

AFCSAMYVGDLCGSV,

(SEQ ID NO: 22)

GVLFGLAYFSMVGNW,

(SEQ ID NO: 23)

VVCCSMSYTWTGALITPC,

(SEQ ID NO: 24)

TRVPYFVRAQGLIRA
and

(SEQ ID NO: 25)

TTLLFNILGGWVAAQ;

or fragments thereof comprising at least 7, preferably at least 8, especially at least 9, amino acid residues containing at least one T-cell epitope. Preferably, the HCV vaccine according to the present invention comprises at least three T-cell epitopes, each from a different hotspot epitope, wherein a hotspot epitope is defined as an epitope containing peptide selected from the group consisting of AYAAQGYKVLVLNPSVAAT (SEQ ID NO:5), GEVQWSTATQSFLATCINGVCWTV (SEQ ID NO:7) and HMWNFISGIQYLAGLSTLPGNPA (SEQ ID NO:8). It is furthermore preferred, if the HCV vaccine according to the present invention further comprises at least one epitope from the hotspot epitopes KFPGGGQIVGGVYLLPRRGPRLGVRATRK (SEQ ID NO:3) and DLMGYIP(A/L)VGAPL (SEQ ID NO:6). Preferably, each of the at least three epitopes are selected from the following three groups:

### GYKVLVLNPSVAAT (SEQ ID NO:4), AYAAQGYKVL (SEQ ID NO:26) or AYAAQGYKVLVLNPSVAAT (SEQ ID NO:5);

### CINGVCWTV (SEQ ID NO:27), GEVQWSTATQSFLAT (SEQ ID NO:48) or GEVQVVSTATQSFLATCINGVCWTV (SEQ ID NO:7); and

HMWNFISGIQYLAGLSTLPGNPA (SEQ ID NO:8), MWNFISGIQYLAGLSTLPGN (SEQ ID NO:28), NFISGIQYLAGLSTLPGNPA (SEQ ID NO:49), QYLAGLSTL (SEQ ID NO:50) or HMWNFISGI (SEQ ID NO:51). It is also preferred to further include at least one epitope from the following groups:

| | |
|---|---|
| KFPGGGQIVGGVYLLPRRGPRLGVRATRK, | (SEQ ID NO: 3) |
| KFPGGGQIVGGVYLLPRRGPRL, | (SEQ ID NO: 52) |
| YLLPRRGPRL, | (SEQ ID NO: 29) |
| LPRRGPRL, | (SEQ ID NO: 30) |
| GPRLGVRAT | (SEQ ID NO: 31) |
| or | |
| RLGVRATRK; | (SEQ ID NO: 32) |
| or | |
| DLMGYIPAV, | (SEQ ID NO: 33) |
| GYIPLVGAPL | (SEQ ID NO: 34) |
| or | |
| DLMGYIPLVGAPL; | (SEQ ID NO: 35) |

A preferred HCV vaccine according to the present invention comprises at least two of the following epitopes:

| | |
|---|---|
| KFPGGGQIVGGVYLLPRRGPRLGVRATRK, | (SEQ ID NO: 3) |
| DLMGYIPAV, | (SEQ ID NO: 33) |
| LEDRDRSELSPLLLSTTEW, | (SEQ ID NO: 14) |
| DYPYRLWHYPCTVNFTIFKV, | (SEQ ID NO: 37) |
| GYKVLVLNPSVAAT, | (SEQ ID NO: 4) |
| CINGVCWTV, | (SEQ ID NO: 27) |
| AAWYELTPAETTVRLR, | (SEQ ID NO: 10) |
| YLVAYQATVCARAQAPPPSWD, | (SEQ ID NO: 15) |
| TAYSQQTRGLLG, | (SEQ ID NO: 39) |
| HMWNFISGIQYLAGLSTLPGNPA, | (SEQ ID NO: 8) |
| IGLGKVLVDILAGYGAGVAGALVAFK | (SEQ ID NO: 12) |
| and | |
| SMSYTWTGALITP | (SEQ ID NO: 40) |

Preferably, the HCV vaccine comprises at least four, preferably at least five, at least six, at least eight, or all twelve of these epitopes.

Another preferred HCV vaccine according to the present invention comprises at least two of the following epitopes:

KFPGGGQIVGGVYLLPRRGPRLGVRATRK (SEQ ID NO:3), DYPYRLWHYPCTVNFTIFKV (SEQ ID NO:37), AAWYELTPAETTVRLR (SEQ ID NO:10), TAYSQQTRGLLG (SEQ ID NO:39), HMWNFISGIQYLAGLSTLPGNPA (SEQ ID NO:8), IGLGKVLVDILAGYGAGVAGALVAFK (SEQ ID NO:12) and SMSYTWTGALITP (SEQ ID NO:40). Preferably, this HCV vaccine comprises at least four, at least five, especially all seven of these epitopes.

The present HCV vaccine preferably comprises at least one A2 epitope and at least one DR1 epitope.

The present HCV vaccine preferably comprises at least one DR7 epitope.

The following combination of epitopes is regarded as specifically powerful (at least one from at least three of the groups (1) to (5)):

| | | |
|---|---|---|
| (1) | KFPGGGQIVGGVYLLPRRGPRLGVRATRK | (SEQ ID NO: 3) |
| or | KFPGGGQIVGGVYLLPRRGPRL | (SEQ ID NO: 52) |
| or | YLLPRRGPRLGVEATRK | (SEQ ID NO: 53) |
| or | YLLPRRGPRL | (SEQ ID NO: 29) |
| or | LPRRGPRL | (SEQ ID NO: 30) |
| or, | LPRRGPRLGVRATRK | (SEQ ID NO: 54) |
| or | GPRLGVRATRK | (SEQ ID NO: 55) |
| or | RLGVRATRK | (SEQ ID NO: 32) |
| or | KFPGGYLLPRRGPRLGVRATRK, | (SEQ ID NO: 56) |
| (2) | AYAAQGYKVLVLNPSVAAT | (SEQ ID NO: 5) |
| or | AYAAQGYKVL | (SEQ ID NO: 26) |
| or | AAQGYKVLVLNPSVAAT | (SEQ ID NO: 57) |
| or | KVLVLNPSVAAT | (SEQ ID NO: 58) |
| or | GYKVLVLNPSVAAT | (SEQ ID NO: 4) |
| or | AYAAQGYKVLVLNPSV | (SEQ ID NO: 59) |
| or | AYAAQGYKVLVLNPSVAA | (SEQ ID NO: 60) |
| or | AAQGYKVLVLNPSVA | (SEQ ID NO: 61) |
| or | AYAAQGYKVLPSVAAT | (SEQ ID NO: 62) |
| or | AYAAQGYKVLAAT, | (SEQ ID NO: 63) |
| (3) | DLMGYIP(A/L)VGAPL | (SEQ ID NO: 6) |
| or | DLMGYIPALVGAPL | (SEQ ID NO: 64) |
| or | DLMGYIP(A/L)VG | (SEQ ID NO: 65) |
| or | DLMGYIP(A/L)VGAP | (SEQ ID NO: 66) |
| or | DLMGYIP(A/L)V | (SEQ ID NO: 67) |
| or | DLMGYIPLVGAPL | (SEQ ID NO: 35) |
| or | DLMGYIPLVGA | (SEQ ID NO: 68) |
| or | DLMGYIPLV, | (SEQ ID NO: 69) |
| (4) | GEVQVVSTATQSFLATCINGVCWTV | (SEQ ID NO: 7) |
| or | GEVQVVSTATQSFLAT | (SEQ ID NO: 48) |
| or | CINGVCWTV | (SEQ ID NO: 27) |
| or | VSTATQSFLATCINGVCWTV | (SEQ ID NO: 70) |
| or | TQSFLATCINGVCWTV | (SEQ ID NO: 71) |
| or | GEVQVVSTATQSFLATCING | (SEQ ID NO: 72) |
| or | GEVQVVSTATQSFLAT, | (SEQ ID NO: 48) |
| (5) | HMWNFISGIQYLAGLSTLPGNPA | (SEQ ID NO: 8) |
| or | MWNFISGIQYLAGLSTLPGNPA | (SEQ ID NO: 73) |
| or | HMWNFISGI | (SEQ ID NO: 51) |
| or | MWNFISGIQYLAGLSTLPGN | (SEQ ID NO: 28) |
| or | NFISGIQYLAGLSTLPGN | (SEQ ID NO: 74) |
| or | QYLAGLSTL | (SEQ ID NO: 50) |
| or | HMWNFISGIQYLAGLSTL | (SEQ ID NO: 75) |
| or | HMWNFISGISTLPGNPA | (SEQ ID NO: 76) |
| or | HMWQYLAGLSTLPGNPA | (SEQ ID NO: 77) |
| or | MWNFISGIQYLAGLSTLPGN; | (SEQ ID NO: 28) |

especially a HCV vaccine comprising the epitopes

| | | |
|---|---|---|
| | GYKVLVLNPSVAAT, | (SEQ ID NO: 4) |
| | DLMGYIPAV, | (SEQ ID NO: 33) |
| | CINGVCWTV | (SEQ ID NO: 27) |
| and | HMWNFISGIQYLAGLSTLPGNPA | (SEQ ID NO: 8) |

has been proven to be specifically powerful.

As mentioned above, the vaccine to be administered bi-weekly according to the present invention comprises a mixture (“pool”) of more than a single antigen. Preferably, the vaccine contains at least three, preferably at least four, especially at least five different HCV T-cell antigens. In other embodiments or if e.g. a larger scope of population should be vaccinated, the mixture may contain 5 to 20, preferably 8 to 15, different (i.e. with a differing amino acid sequence) epitopes.

The amount of peptide antigen proposed for injection has proven to be effective within the range of previously published doses. Accordingly, preferred doses of the HCV vaccine according to the present invention contains—for a pool of peptides as a total amount—from 1 to 20 mg, preferably 3 to 10 mg, especially 4 to 6 mg, HCV T-cell antigens per administration dose.

As mentioned above, the route of administration has also turned out to be of importance for optimising efficacy. The routes having been reported to be efficient for T-cell vaccine administration are also applicable for the present invention. Preferably, the HCV vaccine according to the present invention is administered bi-weekly subcutaneously or intracutaneously, especially intracutaneously (the terms tcrms intradermal (i.d.) and intracutaneous (i.c.) are used interchangeably in the present specification).

The HCV vaccines according to the present invention may contain further immunostimulatory compounds for further stimulating the immune response to the HCV antigen(s). Preferably the further immunostimulatory compound in the pharmaceutical preparation according to the present invention is selected from the group of immunostimulatory deoxynucleotides, alumn, Freund's complete adjuvans, Freund's incomplete adjuvans, immune response modifiers, neuroactive compounds, especially human growth hormone, or combinations thereof. Immunostimulatory deoxynucleotides are e.g. natural or artificial CpG containing DNA, short stretches of DNA derived from non-vertebrates or in form of short oligonucleotides (ODNs) containing non-methylated cytosine-guanine di-nucleotides (CpG) in a certain base context but also inosine and/or uridine containing ODNs (I-ODNs, U-ODNs) as described in WO 01/93905 and WO 02/095027. Neuroactive compounds, e.g. combined with polycationic substances, are described in WO 01/24822.

Within the course of the present invention it turned out that superior results may be obtained if the HCV vaccine according to the present invention is administered in combination with an immune response modifier, preferably with a toll like receptor (TLR) agonist or ligand, especially a toll like receptor (TLR) 7 agonist. Immune response modifiers (IRMs), are a class of unique synthetic molecules that selectively activate toll-like receptors (TLRs), which are critical for stimulating innate and cell-mediated immunity. They have a broad range of potential clinical applications including enhancement of the immune response to vaccine antigens as well as disease-specific monotherapy. The unique TLR activation profiles of IRMs (e.g. TLR 3, TLR7, TLR8 or TLR7 and 8, TLR 9) result in a selective degree of stimulation of various cytokines such as interferon (IFN)-alpha, interleukin-12, IFN-gamma and tumour necrosis factor-alpha. A range of cytokines induced by IRMs enhances cell-mediated immunity and directs it towards a Th1 response which highlights their potential for use as vaccine adjuvants. IRMs are disclosed, e.g., in U.S. Pat. No. 4,689,338, U.S. Pat. No. 5,238,944, U.S. Pat. No. 6,083,505, US 2004/0076633, WO 03/080114 and WO 2005/025583.

Preferably, the HCV vaccine is administered in combination with 1-(2-methylpropyl)-1H-imidazo[4,5-c]quinolin-4-amine (imiquimod), preferably as a topically applied preparation, especially as a cream. An example of such an imiquimod containing cream is commercially available under Aldara™.

Aldara™ is the brand name for an imiquimod containing cream. Each gram of the 5% cream contains 50 mg of imiquimod in an off-white oil-in-water vanishing cream base consisting of isostearic acid, cetyl alcohol, stearyl alcohol, white petrolatum, polysorbate 60, sorbitan monostearate, glycerin, xanthan gum, purified water, benzyl alcohol, methylparaben, and propylparaben.

According to a preferred embodiment of the present invention, the HCV vaccine according to the present invention is administered subcutaneously or intracutaneously (especially intracutaneously) and imiquimod is applied as a cream, preferably as a 5 weight-% cream, directly over the injection site. Imiquimod (Aldara™), as the first commercially available IRM molecule, is approved for the treatment of the viral condition, external genital and perianal warts. Further indications include actinic keratosis and basal cell carcinomas. IRMs, especially Imiquimod, appear to activate Langerhans cells and enhance their migration to lymph nodes. Very recently, imiquimod has also been investigated as an adjuvant for melanoma peptide vaccination in a human trial.

Preferably, for enhancing the immunogenicity of the HCV vaccine according to the present invention, the cream may be applied directly over the injection site (approx. 3×3 cm=9 cm²) after every vaccination, and the injection site may be cleaned gently after a minimum of 8 h. Such a cream may also be applied some time after the injection, e.g. after 4 to 24 hours, preferably 6 to 18 hours, especially 10 to 16 hours, after the initial injection. Alternatively the cream may be applied prior vaccination e.g. 24 hours prior vaccination.

According to another aspect, the present invention relates to the use of at least one HCV T-cell antigen and a polycationic compound comprising peptide bonds for the preparation of an HCV vaccine for treating and preventing HCV infections for a bi-weekly administration of at least 3 times.

Another aspect of the present invention relates to a kit for treating and preventing HCV infections comprising at least four doses of an HCV vaccine as defined herein and an administration tool for a bi-weekly administration.

Preferably, the kit according to the present invention further comprises an immune response modifier as defined herein.

The kit according to the present invention is specifically designed for the bi-weekly administration. Therefore, it preferably contains also means (tools) for assistance for the patient or the medical personnel responsible for bi-weekly administration, such as an administration leaflet for bi-weekly administration, a calendar for bi-weekly administration, an electronic alert dater with a bi-weekly alarm function, or combinations thereof.

### BRIEF DESCRIPTION OF THE DRAWINGS

The invention is further described in the following examples and the drawing figures, yet without being restricted thereto.

FIG. 1 shows that in HLA-A*0201 transgenic mice intradermal application of the HCV vaccine induced stronger HCV peptide-specific T cell responses compared to subcutaneous injection, this response could be further improved by co-application of Aldara™ (immunostimulatory agent: Imiquimod).

FIGS. 2 and 3 show that in HLA-A*0201 transgenic mice increased number of injections augmented the HCV peptide-specific immune response and that the application of an additional immunostimulatory agent gives a faster and more pronounced response against certain HCV-specific MHC class I-restricted epitopes (CD8⁺ T cell responses).

FIG. 4 shows that in HLA-A*0201 transgenic mice injection intervals had an influence on the short term response and that the co-application of an additional immunostimulatory agent induced a sustained response against certain HCV-specific MHC class I-restricted epitopes.

FIG. 5 shows clinical study designs according to examples 5 to 7.

FIG. 6 shows time course of interferon-gamma ELIspot responses to IC41 vaccination applying an optimized schedule. The median of the total ELIspot response (CD4 and CD 8 T cells) (A) and CD8 T cell response (B) among responders in the 5 treatment groups is shown (for calculation of Sum of Vaccine and Sum of Class I see Examples 5 to 7). (C) Critical CD8+ class I T cell response applying optimized and old schedules. Median sum of class I among all ELIspot class I responders are shown.

### DETAILED DESCRIPTION

### Examples

### Example 1

### Influence of the Application Site on the HCV-Peptide-Specific T Cell Response in HLA-A*0201 Transgenic Mice

### Mice HLA-A*0201 Transgenic Mice (HHD.2)

Vaccine: clinical batch PD03127 (lot K)
Injection volume of 100 μl per mouse contains:

| | | |
|---|---|---|
| As antigens: | | |
| Ipep 83 | | |
| (KFPGGGQIVGGVYLLPRRGPRL | (SEQ ID NO: 52)) | 200 μg, |
| Ipep 84 | | |
| (GYKVLVLNPSVAAT | (SEQ ID NO: 4)) | 200 μg, |
| Ipep 87 | | |
| (DLMGYIPAV | (SEQ ID NO: 33)) | 200 μg, |
| Ipep 89 | | |
| (CINGVCWTV | (SEQ ID NO: 27)) | 200 μg, |
| Ipep 1426 | | |
| (HMWNFISGIQYLAGLSTLPGNPA | (SEQ ID NO: 8)) | 200 μg |

As adjuvant: Poly-L-Arginine with an average degree of polymerisation of 40 to 50 arginine residues (determined by multiple angle laser light scattering (MALLS)); lot 113K7277; Sigma Aldrich Inc.; 400 μg
Additional adjuvant: Aldara™ containing 5% Imiquimod, an immunostimulatory agent acting via TLR7; 3M Health Care Ltd.; dose: approx 20 mg/mouse
Formulation buffer: 5 mM phosphate/270 mM sorbitol
   Experimental set-up 10 mice per group
   1. subcutaneous injection into the flank
   2. intradermal injection into the back
   3. intradermal injection into the back followed by immediate application of Aldara™ cream at injection area

On days 0, 14 and 28 mice were injected with a total amount of 100 μl/vaccine/mouse containing the above listed compounds at different sites as indicated. Spleens were harvested for each experimental group on day 35 and enriched for CD4⁺ T cells by magnetic separation (MACS). CD4⁺ T cell-depleted spleen cells were used to determine the CD8⁺ T cell response. MHC class II restricted (CD4⁺ T cells) as well as MHC class I restricted T cell responses (CD8⁺ T cells) against each single HCV-derived peptide were determined using an IFN-7 ELIspot assay. In general, restimulation with an irrelevant peptide induced no IFN-7 production.

### Results

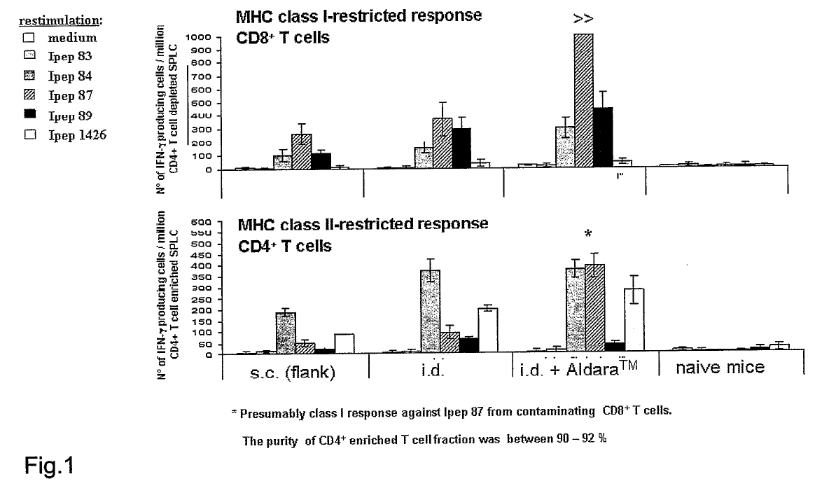

As shown in FIG. 1, upon subcutaneous injection MHC class I-restricted CD8⁺ T cell responses could be detected against Ipeps 84, 87 and 89, and MHC class II-restricted CD4⁺ T cell responses against Ipeps 84 and 1426. These responses could be further augmented by intradermal application of the vaccine. Moreover, co-application of Aldara™ directly after intradermal injection further increased the detected responses, especially the MHC class I-restricted CD8⁺ T cell response against Ipep 87.

In conclusion, intradermal application of the HCV vaccine induced stronger HCV peptide-specific T cell responses compared to subcutaneous injection, this response could be further improved by co-application of Aldara™.

### Example 2

### HCV-Peptide-Specific MHC Class I-Restricted CD8+T Cell Responses Upon Single, Two or Three Injections in HLA-A*0201 Transgenic Mice

### Mice HLA-A*0201 Transgenic Mice (HHD.2)

Vaccine: Injection volume of 100 μl per mouse contains:
As antigens: Ipep 83 200 μg, Ipep 84 200 μg, Ipep 87 200 μg, Ipep 89 200 μg, Ipep 1426 200 μg
As adjuvant: Poly-L-Arginine with an average degree of polymerization of 40 to 50 arginine residues (determined by MALLS); lot 113K7277; Sigma Aldrich Inc.; 400 μg
Additional adjuvant: Aldara™ containing 5% Imiquimod, an immunostimulatory agent acting via TLR7; 3M Health Care Ltd.; dose: approx 20 mg/mouse
Formulation buffer: 5 mM phosphate/270 mM sorbitol
   Experimental set-up 30 mice per group (10 per time point of analysis)
   1. intradermal injection into the back
   2. intradermal injection into the back followed by immediate application of Aldara™ cream at injection area

On days 0, 14 and 28 mice were injected intradermally with a total amount of 100 μl/vaccine/mouse containing the above listed compounds. Spleens were harvested for each experimental group on days 7, 21 and 35 and depleted for CD4⁺ T cells by magnetic separation (MACS). IFN-γ production by MHC class I-restricted CD8⁺ T cells upon re-stimulation with single HCV-derived peptides was determined by ELISpot assay. In general, restimulation with an irrelevant peptide induced no IFN-γ production.

In addition, an in vivo CTL assay was performed to determine the effector function of MHC class I-restricted CD8⁺ T cells upon single or booster injection. In brief, antigen-presenting cells (APC) prepared from naïve mice were either loaded with Ipep 87 and labeled with CFSE^{high} or, for control purposes, loaded with Ipep1247 (irrelevant peptide) and labeled with CFSE^{medium} or without peptide loading labeled with CFSE^{low}. These APC were mixed together (1:1:1) and adoptively transferred via i.v. injection into vaccinated mice at days 6, 20 or 34. One day later (days 7, 21 or 35), FACS analyses were performed in order to detect the absence (indicating a vaccination-induced killing) or the presence of transferred APC loaded with relevant peptide. No killing of unloaded APC was observed in any experiment.

### Results

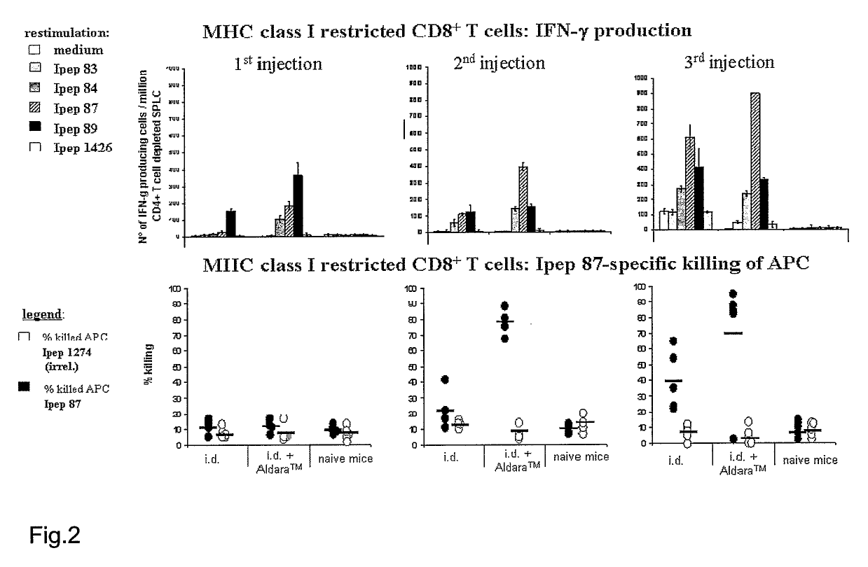

As shown in FIG. 2 upper graphs, HCV peptide-specific IFN-7 production by MHC class I-restricted CD8+T cells was detectable upon single or booster intradermal injections differing in regard to the strength of the response to certain peptides.

In detail, upon single intradermal injection a response was detectable only against Ipep 89, whereas upon two injections a response against Ipep 84, 87 and 89 was induced in comparable strength. This response was further augmented by a third injection clearly showing a dominance of the response against Ipep87 over those against Ipeps 89 and 84.

In contrast, the co-application of Aldara™ induced a response against all three peptides already upon single injection. Upon 2^{nd} application the pre-dominant response against Ipep 87 could be already seen. The third application further increased the strength of the Ipep 87-specific response.

As shown in FIG. 2 lower graphs, two injections were necessary to induce Ipep 87-specific effector function of MHC class I-restricted CD8⁺ T cells. Moreover, the effector function was significant and strongly increased upon co-application of Aldara™.

In summary, the results show that increased number of injections augmented the HCV-specific immune response. In addition, the application of an additional immunostimulatory agent (Aldara™) gave a faster and more pronounced response against certain MHC class I-restricted CD8⁺ T cell epitopes.

### Example 3

### HCV-Peptide-Specific MHC Class I-Restricted CD8⁺ T Cell Responses Upon Three or Six Injections in HLA-A*0201 Transgenic Mice

### Mice HLA-A*0201 Transgenic Mice (HHD.2)

Vaccine clinical batch PD03127 (lot K)
Injection volume of 100 μl per mouse contains:
As antigens: Ipep 83 200 μg, Ipep 84 200 μg, Ipep 87 200 μg, Ipep 89 200 μg, Ipep 1426 200 μg
As adjuvant: Poly-L-Arginine with an average degree of polymerization of 40 to 50 arginine residues (determined by MALLS); lot 113K7277; Sigma Aldrich Inc.; 400 μg
Additional adjuvant: Aldara™ containing 5% Imiquimod, an immunostimulatory agent acting via TLR7; 3M Health Care Ltd.; dose: approx 20 mg/mouse
   Formulation buffer 5 mM phosphate/270 mM sorbitol
   Experimental set-up 20 mice per group (10 per time point of analysis)
   1. subcutaneous injection into the flank
   2. intradermal injection into the back
   3. intradermal injection into the back followed by immediate application of Aldara™ cream at injection area

On days 0, 14, 28, 43, 58 and 71 mice were injected with a total amount of 100 μl/vaccine/mouse containing the above listed compounds at different sites as indicated. Spleens were harvested for each experimental group on day 35 or day 78 and depleted for CD4⁺ T cells by magnetic separation (MACS). IFN-γ production by MHC class I-restricted CD8⁺ T cells upon re-stimulation with single HCV-derived peptides was determined by ELISpot assay. In general, restimulation with an irrelevant peptide induced no IFN-γ production.

### Results

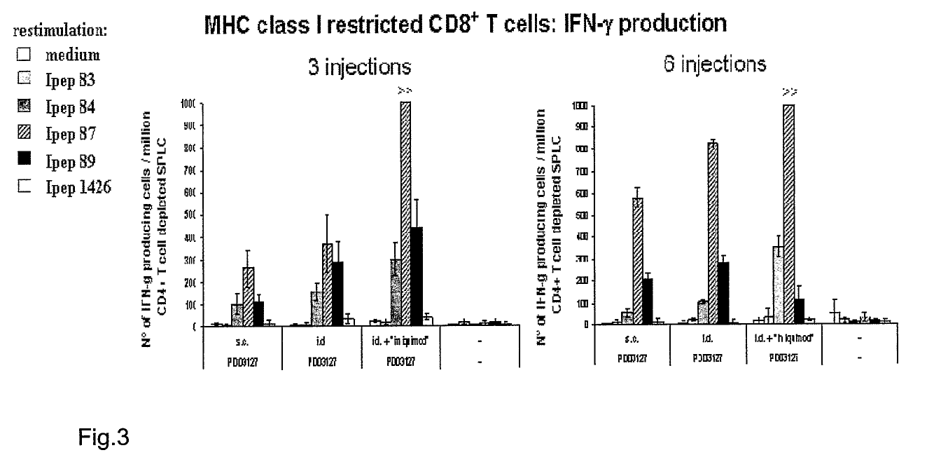

FIG. 3 shows IFN-γ production by MHC class I-restricted CD8⁺ T cells obtained upon six versus three injections. Independent of the application site, the response especially against Ipep 87 could further be enhanced by additional vaccinations. The strongest response was always seen upon co-application of vaccine and Aldara™.

In summary, the data show that increased number of injections augmented the HCV-specific immune response. In addition, the application of an additional immunostimulatory agent (Aldara™) gave more pronounced responses against certain MHC class I-restricted CD8⁺ T cell epitopes.

### Example 4

### Short and Long Term HCV-Peptide-Specific MHC Class I-Restricted CD8⁺ T Cell Responses in HLA-A*0201 Transgenic Mice Upon Three Injections Based on Different Injection Intervals

### Mice HLA-A*0201 Transgenic Mice (HHD.2)

Vaccine: Injection volume of 100 μl per mouse contains:
As antigens: Ipep 83 200 μg, Ipep 84 200 μg, Ipep 87 200 μg, Ipep 89 200 μg, Ipep 1426 200 μg
As adjuvant: Poly-L-Arginine with an average degree of polymerization of 40 to 50 arginine residues (determined by MALLS); lot 114K7276; Sigma Aldrich Inc.; 400 μg
Additional adjuvant: Aldara™ containing 5% Imiquimod, an immunostimulatory agent acting via TLR7; 3M Health Care Ltd.; dose: approx 20 mg/mouse
   Formulation buffer 5 mM phosphate/270 mM sorbitol
   Experimental set-up 20 mice per group (10 per time point of analysis)
   1. subcutaneous injection into the flank
   2. intradermal injection into the back
   3. intradermal injection into the back followed by immediate application of Aldara™ cream at injection area

Mice were injected three times based on 1-week, 2-week or 4-week interval with a total amount of 10 μl/vaccine/mouse containing the above listed compounds at different sites as indicated. Spleens were harvested for each experimental group on day 7 and day 110 after third injection and depleted for CD4⁺ T cells by magnetic separation (IACS). Upon re-stimulation with single HCV-derived peptides, IFN-γ production by MHC class I-restricted CD8⁺ T cells was determined by ELISpot assay. In general, restimulation with an irrelevant peptide induced no IFN-γ production.

### Results

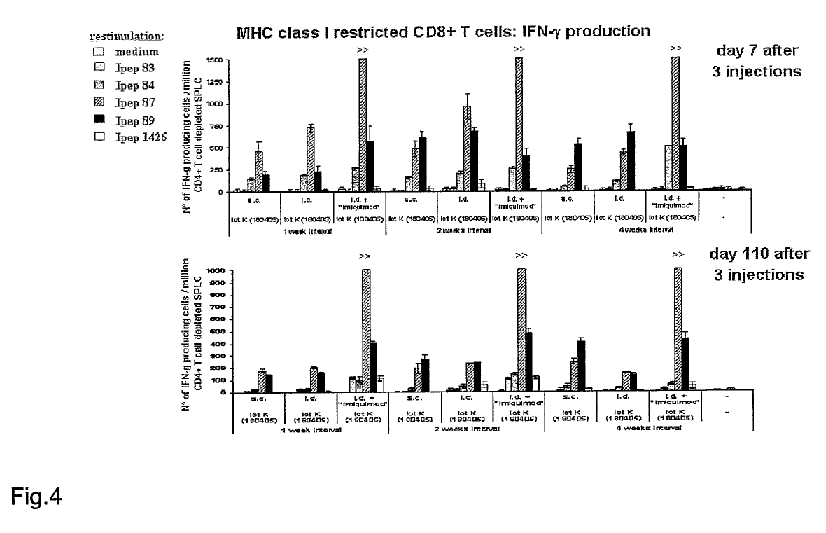

As shown in FIG. 4 upper graph, a slightly stronger MHC class I-restricted CD8⁺ T cell response was seen upon subcutaneous or intradermal 2-week injection interval compared to 1- or 4-week injection intervals at the respective application sites. No significant difference regarding the influence of injection intervals was seen upon co-application of vaccine and Aldara™.

FIG. 4 lower graphs show that the different injection intervals had no influence on the persistence of HCV peptide-specific MHC class I-restricted CD8⁺ T cell responses. However, the data clearly indicate a superior induction of Ipep 87- and Ipep 89-specific MHC class I-restricted CD8⁺ T cell responses upon co-application of Aldara™ compared to intradermal or subcutaneous injection of the vaccine alone.

In summary, it is shown that injection intervals have an influence on the short term response and co-application of an additional immunostimulatory agent (Aldara™) induced a very sustained response against certain HCV-specific MHC class I-restricted epitopes.

### Examples 5 to 7

### Clinical Trials

Clinical trials have been performed with a pool of HCV T-cell antigens (the vaccine is termed “IC41” and consists of a mixture of synthetic peptides representing conserved T cell epitopes of HCV plus Poly-L-Arginine as a synthetic T cell adjuvant; IC 41 comprises five peptides from different regions from the HCV polypeptide, i.a. the following three epitopes: HMWNFIS-GIQYLAGLSTLPGNPA (SEQ ID NO:8), CINGVCWTV (SEQ ID NO:27) and DLMGYIPAV (SEQ ID NO:33)). IC41 therefore contains 5 synthetic peptides mainly derived from the nonstructural regions NS3 and NS4 which are known to be targets of productive immune responses in patients. They harbor at least 4 HLA-A*0201 restricted CTL-epitopes and 3 highly promiscuous CD4+Helper T cell epitopes and all of these have been shown to be targeted in patients responding to standard treatment or spontaneously recovering from HCV. With one exception peptide sequences are highly conserved in genotype 1. IC41 contains poly-L-Arginine as synthetic adjuvant, which has been shown to augment Th1/Tc1 (IFN-γ) responses in animal studies. Data from clinical with IC41 showed that administration of the vaccine is safe and well-tolerated and that IC41 can induce HCV-specific Th1/Tc1-responses in healthy volunteers, as well as in chronic HCV patients.

As read-out for vaccine immunogenicity validated T cell assays (Interferon-gamma ELIspot Assay, T cell Proliferation Assay, HLA-tetramer/FACS assay) were used as described. These assays allow reliable measurements of epitope-specific T cell responses induced by the therapeutic HCV vaccine IC41. The vaccine-induced T cell immune responses serve as surrogate parameters of efficacy. ELIspot allows quantification of peptide-specific, functional (i.e. cytokine-secreting) T cells in biological samples like human blood. The basis of the assay is that, T cells upon stimulation with a peptide specifically recognized by the T cell receptor react by secretion of cytokines like IFN-γ. This reaction can be carried out in a 96-well plate. The filter-wells of this plate are coated with a Mab specific for IFN-y. Consequently, each cell secreting IFN-γ leaves an IFN-γ spot, which can be visualized with a subsequent color reaction. Spots can be counted using automated plate readers. Numbers obtained are a measure for the frequency of peptide-specific, IFN-γ-secreting T cells in the sample. ELIspot was done individually for each of the 5 peptides of IC41, in addition, 3 HLA-A2 epitopes contained within longer peptides were tested individually.

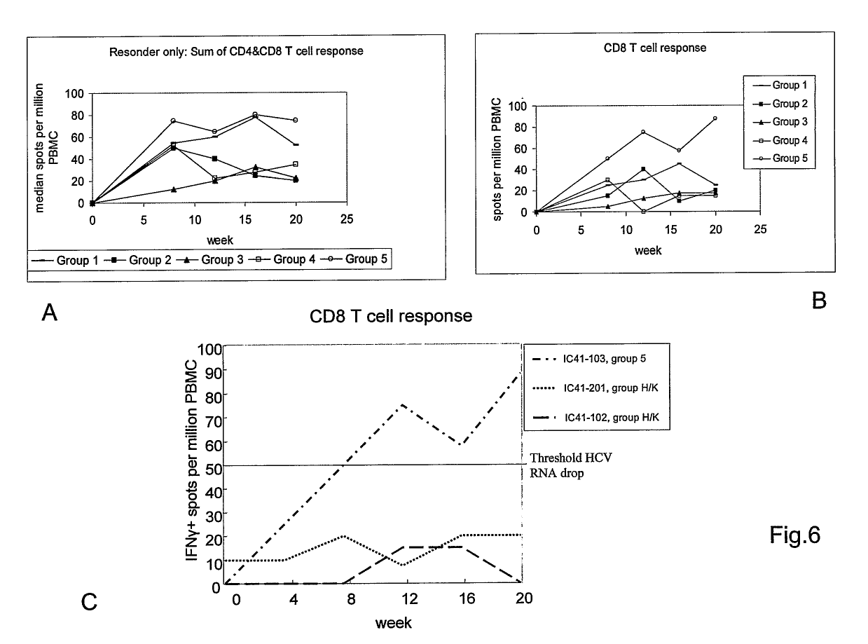

Use of an external standard on each ELIspot assay plate in the clinical trials IC41-102 (healthy volunteers), IC41-201 (chronic non responder patients, PCT/EP2005/054773) and IC41-103 (application optimization in healthy volunteers) allow a direct comparison of data from these trials (the designs of clinical studies IC41-102, IC41-201 and IC41-103 are shown in FIG. 6).

### Example 5

### Responder Rates Improved

Response was scored if any peptide tested, at any time-point during or after vaccination was at least 3-fold above the baseline value or at least significantly positive if baseline was zero.

All groups in IC41-103 showed an improved response rate as compared to the 4 times every 4 week schedule applied in IC41-102. Highest responder rate for CD8+ T cell responses was achieved in group 3 with the most frequent (weekly) schedule. A possible explanation is an at least partially CD4+ T helper cell independent CD8+ T cell activation through the intense and frequent vaccination stimulus.

TABLE 1
ELIspot Responder Rates in Groups 1-5 in study
IC41-103 as compared to Group K, IC41-102

| | | | CD8+ | CD4+ | | |
|---|---|---|---|---|---|---|
| Group | N analyzed | NRE | (REIV) | (REV) | % CD8 | % CD4 |
| 1 | 8 | 0 | 7 | 8 | 88% | 100% |
| 2 | 7 | 2 | 5 | 5 | 71% | 71% |
| 3 | 8 | 0 | 8 | 5 | 100% | 63% |
| 4 | 9 | 1 | 7 | 7 | 78% | 78% |
| 5 | 9 | 1 | 6 | 8 | 67% | 89% |
| 102-K | 12 | 6 | 5 | 6 | 42% | 50% |

(NRE: non-responders in ELIspot, REIV: CD8+ T cell ELIspot Responders, REV: CD4+ T cell Responders)

### Example 6

### Sum of Vaccine & Sum of Class I ELIspot Improved

To assess quantitatively the IFN-gamma T cell response evoked by IC41 vaccination, time courses of ELIspot responses for each individual were determined: Sum of vaccine was calculated by adding up ELIspots measured individually against each of the five peptides of IC41 after subtraction of background (irrelevant HIV peptide subtracted). Sum of Class I was calculated by adding up ELIspots measured individually against each of the five HLA-A2 epitopes of IC41 after subtraction of background (irrelevant HIV peptide subtracted). The maximum sum of vaccine and maximum sum of class I (both usually recorded after the last vaccination) was determined and the median values over all responders per group were determined (see Table 2).

TABLE 2
Total ELIspots elicited through IC41.

| | | | Median of Sum of Class | |
|---|---|---|---|---|
| | Median of Sum of | | I (CD8+ T cells) over | |
| | Vaccine over all | | all Class I ELIspot | |
| | ELIspot responders | | responders | |
| IC41 treatment | (spots per Mio. PBMC) | | (spots per Mio. PBMC) | |
| ALL IC41-102 | 35-40 | (n = 37) | 20 | (n = 23) |
| & -201 verum | | | | |
| groups | | | | |
| Group 1 (n = 8) | 100 | (n = 8) | 45 | (n = 7) |
| Group 2 (n = 7) | 60 | (n = 5) | 50 | (n = 5) |
| Group 3 (n = 8) | 45 | (n = 8) | 35 | (n = 8) |
| Group 4 (n = 9) | 90 | (n = 8) | 60 | (n = 7) |
| Group 5 (n = 9) | 95 | (n = 8) | 105 | (n = 6) |

For determination of Sum Vaccine and Sum Class I see text, n specifies number of ELIspot responders.
In IC41-201 an association of a type I (IFN-gamma), CD8+ T cell response and decline of HCV RNA was observed in several patients: data available suggested that a threshold level of at least 50 CD8+ T cell ELIspots/million PBMC were required for a rapid greater one log10 decrease of HCV RNA. Therefore, one aim of the optimization study was to achieve this level of immunogenicity in at least a subset of vaccines.

As shown in Table 2, the median sum class I in ELIspot class I responders in IC41-103 groups 1, 2 and 4 reached this threshold, whereas group 3 and all responders treated with the old 4 (IC41-102) or 6 times (IC41-201) every 4 week schedule did not. Clearly IC41-103 group 5 was best in achieving more than double of the required threshold.

The time course of the median sum vaccine and median sum class I for groups 1 to 5 is shown in FIGS. 6A and B. The dramatic increase in the critical CD8+ class I T cell response as compared to the old 4 (IC41-102) or 6 times (IC41-201) every 4 week schedule is shown in FIG. 6C.

### Example 7

Breadth of Critical Class I (CD8+) T cell Response Improved

In order to prevent escape mechanisms like mutational epitope escape, another goal was to achieve a broad response, i.e. simultaneous T cell responses against more than one class I epitope in the same individual at the same time. In the two studies concluded before that applied the old 4 (IC41-102) or 6 times (IC41-201) every 4 week schedule, at best one dominant CD8+T cell epitope induced a response.

In order to compare the breadth of class I responses, the median number of CD8+ T cell epitopes raising responses within one subject was determined among all ELIspot class I responders per group (see Table 3).

As shown in Table 3, median number of CD8+ T cell epitopes giving rise to response within a subject could be doubled in IC41-103 groups 1, 3 and 4. Again, IC41-103 group 5 was best achieving a median of 3 (out of 5 possible) CD8+ T cell epitopes targeted simultaneously within a subject.

TABLE 3
Breadth of critical class I (CD8+) T cell response.

| | | Median number of |
|---|---|---|
| | | CD8+ cytotoxic T cell epitopes |
| IC41 treatment | N total | within one subject |
| ALL IC41-102 & -201 | 120 | 1 (n = 23) |
| verum groups | | |
| Group 1 | 8 | 2 (n = 7) |
| Group 2 | 7 | 1 (n = 5) |
| Group 3 | 8 | 2 (n = 8) |
| Group 4 | 9 | 2 (n = 7) |
| Group 5 | 9 | 3 (n = 6) |

N total specifies the number of subjects/patients treated, n specifies number of ELIspot class I responders.

These clinical and preclinical results show that the optimal application of IC41 in terms of strength and breadth (see Tab 2 and 3) of interferon-gamma ELIspot response was identified to be group 5 in an injection interval of most preferably 2 weeks. 1 week was weaker than bi-weekly; 4 weeks was clearly worse. Intracutaneous (intradermal) treatment was slightly superior to subcutaneous treatment (no difference between groups 1 (s.c.) and 4 i.d.) but best result group 5. The topical application of Aldara™/imiquimod, a toll-like receptor 7 agonist resulted in an improvement of the clinical results.

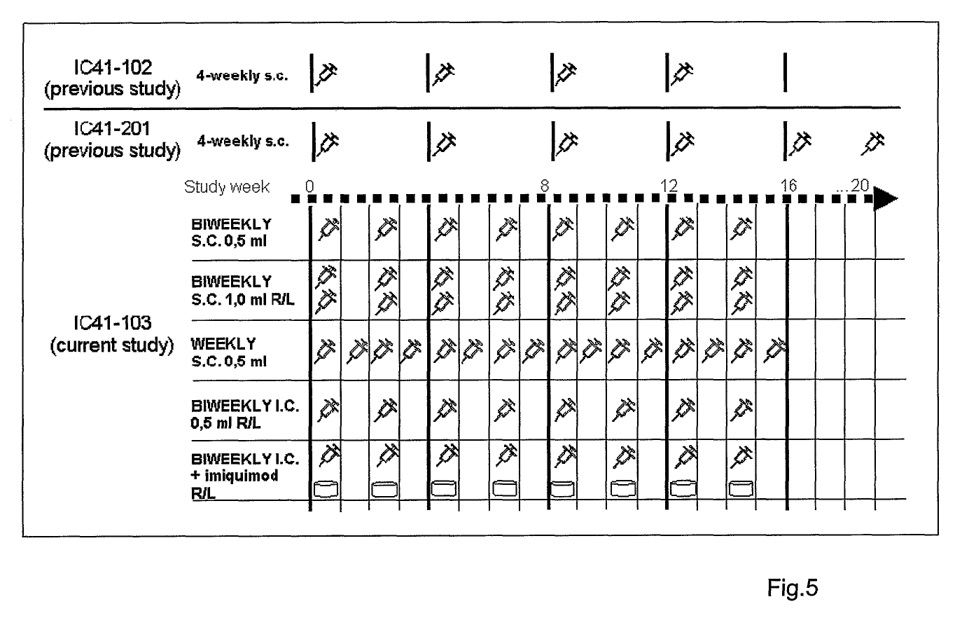

Group 3 (weekly, s.c.), shows 100% CD8+ T cell responders but only 63% CD4+ T cell responses (Tab 1 responder rates). This is interpreted as CD4+ independent activation of CD8+ T cells through frequent (weekly) application. A comparison of Groups 1 (s.c.) and 4 (i.d.) suggests that there is no significant difference regarding route. It is also shown that the absolute number of injections does not seem to increase strength (Tab 2: 16 vaccinations in groups 2 and 3 vs. 8 vaccinations in other groups) plus FIG. 5 top: plateau already at week 8=after 4 (or 8 in Groups 2 and 3) vaccinations). Finally the breadth of CD8+ response=simultaneous response against several class I epitopes within individual subject/patient (requires processing of “hotspot” peptides (WO 2004/024182) that contain minimal class I epitope within larger sequence) works best in Group 5.

1.-19. (canceled)

20. A method for preventing or treating Hepatitis C Virus (HCV) infections comprising:
obtaining a composition comprising an effective amount of at least one HCV T-cell antigen and a polycationic compound comprising peptide bonds; and
administering the compositions to a human individual bi-weekly at least 3 times.

21. The method of claim 20, wherein the composition is administered bi-weekly at least 4 times.

22. The method of claim 21, wherein the composition is administered bi-weekly at least 6 times.

23. The method of claim 22, wherein the composition is administered bi-weekly at least 8 times.

24. The method of claim 20, wherein the polycationic compound comprising peptide bonds comprises a basic polypeptide, organic polycation, or basic polyamino acid.

25. The method of claim 20, wherein the polycationic compound comprising peptide bonds comprises a peptide chain having a chain length of at least 4 amino acid residues.

26. The method of claim 20, wherein the polycationic compound comprising peptide bonds is a polypeptide containing more than 20% basic amino acids in a range of more than 8 amino acid residues.

27. The method of claim 26, wherein the polycationic compound comprising peptide bonds is a polypeptide containing more than 20% basic amino acids in a range of more than 20 amino acid residues.

28. The method of claim 26, wherein the polycationic compound comprising peptide bonds is a polypeptide containing more than 50% basic amino acids in a range of more than 8 amino acid residues.

29. The method of claim 28, wherein the polycationic compound comprising peptide bonds is a polypeptide containing more than 50% basic amino acids in a range of more than 20 amino acid residues.

30. The method of claim 26, wherein the polycationic compound comprising peptide bonds is polyarginine, polylysine, a polycationic antimicrobial peptide, or a peptide containing at least 2 KLK-motifs separated by a linker of 3 to 7 hydrophobic amino acids.

31. The method of claim 20, wherein the polycationic compound comprising peptide bonds contains between 20 and 500 amino acid residues.

32. The method of claim 31, wherein the polycationic compound comprising peptide bonds contains between 30 and 200 residues.

33. The method of claim 20, wherein the HCV T-cell antigen is a polypeptide consisting of from 7 to 50 amino acid residues and comprising at least one T-cell epitope.

34. The method of claim 33, wherein the HCV T-cell antigen is a polypeptide consisting of from 8 to 45 amino acid residues and comprising at least one T-cell epitope.

35. The method of claim 34, wherein the HCV T-cell antigen is a polypeptide consisting of from 8 to 20 amino acid residues and comprising at least one T-cell epitope.

36. The method of claim 20, wherein the HCV T-cell antigen is selected from one or more of:
(SEQ ID NO: 3)
KEPGGGQIVGGVYLLPRRGPRLGVRATRK;
(SEQ ID NO: 4)
GYKVLVLNPSVAAT;
(SEQ ID NO: 5)
AYAAQGYKVLVLNPSVAAT;
(SEQ ID NO: 6)
DLMGYIP(A/L)VGAPL;
(SEQ ID NO: 7)
GEVQVVSTATQSFLATCINGVCWTV;
(SEQ ID NO: 8)
HMWNFISGIQYLAGLSTLPGNPA;
(SEQ ID NO: 9)
VDYPYRLWHYPCT(V/I)N(F/Y)TIFK(V/I)RMYVGGVEHRL;
(SEQ ID NO: 10)
AAWYELTPAETTVRLR;
(SEQ ID NO: 11)
GQGWRLLAPITAYSQQTRGLLGCIV;
(SEQ ID NO: 12)
IGLGKVLVDILAGYGAGVAGALVAFK;
(SEQ ID NO: 13)
FTDNSSPPAVPQTFQV;
(SEQ ID NO: 14)
LEDRDRSELSPLLLSTTEW;
(SEQ ID NO: 15)
YLVAYQATVCARAQAPPPSWD;
(SEQ ID NO: 16)
MSTNPKPQRKTKRNTNR;
(SEQ ID NO: 17)
LINTNGSWHINRTALNCNDSL;
(SEQ ID NO: 18)
TTILGIGTVLDQAET;
(SEQ ID NO: 19)
FDS(S/V)VLCECYDAG(A/C)AWYE;
(SEQ ID NO: 20)
ARLIVFPDLGVRVCEKMALY;
(SEQ ID NO: 21)
AFCSAMYVGDLCGSV;
(SEQ ID NO: 22)
GVLFGLAYFSMVGNW;
(SEQ ID NO: 23)
VVCCSMSYTWTGALITPC;
(SEQ ID NO: 24)
TRVPYFVRAQGLIRA;
(SEQ ID NO: 25)
TTLLFNILGGWVAAQ;
or a fragment of one of these comprising at least 7 amino acid residues and containing at least one T-cell epitope.

37. The method of claim 36, wherein the HCV T-cell antigen is a fragment of one of the listed sequences comprising at least 8 amino acid residues and containing at least one T-cell epitope.

38. The method of claim 37, wherein the HCV T-cell antigen is a fragment of one of the listed sequences comprising at least 8 amino acid residues and containing at least one T-cell epitope.

39. The method of claim 20, wherein the composition comprises a mixture of at least three different HCV T-cell antigens.

40. The method of claim 39, wherein the composition comprises a mixture of at least four different HCV T-cell antigens.

41. The method of claim 40, wherein the composition comprises a mixture of at least five different HCV T-cell antigens.

42. The method of claim 39, wherein the composition contains from 1 to 20 mg HCV T-cell antigens per administration dose.

43. The method of claim 42, wherein the composition contains from 3 to 10 mg HCV T-cell antigens per administration dose.

44. The method of claim 43, wherein the composition contains from 4 to 6 mg HCV T-cell antigens per administration dose.

45. The method of claim 42, wherein the composition is administered subcutaneously or intracutaneously.

46. The method of claim 45, wherein the composition is administered intracutaneously.

47. The method of claim 45, wherein the composition is administered in combination with an immune response modifier.

48. The method of claim 47, wherein the immune response modifier is a toll like receptor (TLR) agonist.

49. The method of claim 48, wherein the immune response modifier is a toll like receptor (TLR) 7 agonist, a TLR 7 and 8 agonist, and/or a TLR 9 agonist.

50. The method of claim 47, wherein the composition is administered in combination with 1-(2-methylpropyl)-1H-imidazo[4,5-c]quinolin-4-amine (imiquimod).

51. The method of claim 50, wherein the composition is administered topically.

52. The method of claim 51, wherein the composition is administered in a cream.

53. The method of claim 50, wherein the composition is administered subcutaneously or intracutaneously and imiquimod is applied directly over the injection site.

54. The method of claim 53, wherein imiquimod is applied as a 5 weight-% cream, directly over the injection site.

55. The method of claim 53, wherein imiquimod is applied 4 to 24 hours after administration of the composition.

56. The method of claim 53, wherein imiquimod is applied 10 to 16 hours after administration of the composition.

57. A kit comprising at least four doses of a composition comprising an effective amount of at least one HCV T-cell antigen and a polycationic compound comprising peptide bonds and an administration tool for a bi-weekly administration.

58. The kit of claim 57, further comprising an immune response modifier.

59. The kit of claim 57, wherein the administration tool is an administration leaflet, a calendar, or an electronic alert dater with a bi-weekly alarm function.
